(19) United States
(12) Patent Application Publication
(10) Pub. No.: US 2011/0217679 A1
(43) Pub. Date: Sep. 8, 2011

(54) DIAGNOSIS METHOD AND SYSTEM BASED ON HANDWRITING ANALYSIS

(75) Inventor: Sara Rosenblum, Mitzpe Hoshaya (IL)

(73) Assignee: CARMEL-HAIFA UNIVERSITY ECONOMIC CORPORATION LTD., Haifa (IL)

(21) Appl. No.: 13/127,649

(22) PCT Filed: Nov. 5, 2009
(86) PCT No.: PCT/IL2009/001034

§ 371 Date: May 4, 2011

Related U.S. Application Data
(60) Provisional application No. 61/111,558, filed on Nov. 5, 2008.

(51) Int. Cl. A61B 5/00; G09B 7/00

(52) U.S. Cl. 434/155; 434/350

(57) ABSTRACT

A diagnosis system and method comprising a handwriting data collection and analysis software and accompanying tools for diagnosing a given condition such as an illness, a mental illness, a physiological condition, a mental or emotional state, medication effect, a personality trait, a skill, an expertise, deception or truth writing. The handwriting of a control group known to have a condition is analyzed in order to determine the detectable characteristic indicators of that group. Such measured indicators comprise spatial, temporal and pressure measures for each writing stroke. The handwriting of a subject is then analyzed to determine if the measured indicators indicate statistically that the subject can be diagnosed with a condition.

### FIELD OF THE INVENTION

The present invention relates to diagnosis based on activity analysis and in particular to conditions, function-dysfunction, abilities and behavior diagnosis based on objective brain-hand performance such as handwriting evaluation and accompanying tools.

### BACKGROUND OF THE INVENTION

The International Classification of Functioning Disability and Health (ICF) is a concept system and reference model published by the World Health Organization (WHO, 2001). According to the WHO, concepts of health and illness are no longer defined exclusively according to body structures and functions but now also relates to how a person performs activities and tasks required from him in every day life and how that person behaves and participates in various environments such as home and family, work, academic and social environments and leisure activities, as shown in FIG. 1.

However, despite of the meaningful change in concepts, there is still lack of evaluation tools in the clinical field in order to evaluate a patient's qualitative and quantitative aspects of activity and participation.

Most available tools are still focused on body functions and structures (e.g., body temperature, blood pressure, weight) or on isolated aspects required for human activity and performance such as metacognition (Executive Functions) memory, gross or fine motor or sensational aspects.

This lack stems from the fact that an entire activity or aspects of participation in activities in everyday life are a complex issue to evaluate or measure in comparison with physical body functions or separate aspects required for performance.

Many known methods aim to diagnose medical conditions such as an illness, a mental illness, a physiological condition or a medication effect. Some drawbacks of such methods include: significant cost of equipment, invasive diagnosis, and complex set-up and diagnosis procedure.

Diagnosing a mental or emotional state, a personality trait, a skill, an expertise, or deception or truth telling through real life activity performance is much more challenging and often not very precise and objective.

There is thus a need to develop a diagnostic method that would overcome the abovementioned drawbacks of current diagnostic methods.

### SUMMARY OF THE INVENTION

Handwriting is a complex human activity that appears to be an outward manifestation of the individual's perceptual-motor abilities. Handwriting is a measurable expression of a person's functional capabilities or state. Collecting objective data regarding people's conditions, abilities and behavior through handwriting performance is important for various purposes such as a personality trait, a skill, an expertise, the classification of people into appropriate professions, legal purposes, deception or truth writing, early detection of disease/dysfunction, an illness, a mental illness, a physiological condition, a mental or emotional state, refinement of certain medical diagnoses and hence prevention, identification of how drugs influence disease processes, educational assessment and intervention, or any combination thereof. A software tool (developed by the inventor) Computerized Penmanship Evaluation Tool (COMPET) enables data collection and sophisticated data analysis of various handwriting tasks. The analysis is based on recorded temporal, spatial and pressure indicators. This data in combination with other evaluation tools developed by the author enables obtaining unique information regarding people's performance in every day life.

The term “handwriting” as referred herein, should be interpreted in a large sense to encompass any writing or drawing activities, for example, writing letters and/or numbers, drawing simple or complex figures or any combination thereof.

As the following detailed description will show, one inventive aspect of the present invention is related to a computerized system and method for diagnosing a human condition. The condition can range from a medical condition (illness, mental illness, reaction to medication) to a mental state (stressed, writing the truth or lying, depressed) to detecting personality traits, skills or expertise.

The present invention includes several inventive aspects, including, by way of non-limiting examples:
analyzing handwriting via detectable indicators captured by a computerized system;
defining for each condition to be diagnosed, a predetermined list of characteristic indicators. The indicators can be obtained by analyzing the handwriting of people known to have that condition (i.e. Alzheimer) and establishing what measures/indicators characterize that group (i.e. people with Alzheimer). Alternatively, the system can analyze a person while not having a condition (i.e. writing a truthful event) and then comparing it to handwriting of the same person when having that condition (i.e. knowingly letting the person write a lie); and
analyzing the handwriting of a person in order to determine if the person's handwriting indicators correlate statistically with the indicators of that condition.

The present invention thus relates to a diagnosis method based on handwriting analysis, the method comprising the steps of:

(i) defining for each condition a list of characteristic indicators detectable while performing a set of predetermined handwriting tasks;

(ii) recording the handwriting tasks of a person according to the defined characteristic indicators of said condition; and

(iii) analyzing the values of the recorded indicators in order to determine whether the person is diagnosed with said condition.

In a preferred embodiment of the present invention the diagnosed condition comprises: an illness, a mental illness, a physiological condition, a mental or emotional state, medication effect, a personality trait, a skill, an expertise, deception or truth writing, or any combination thereof.

In one preferred embodiment of the present invention the characteristic indicators comprise spatial, temporal and pressure measures for each writing stroke.

In another preferred embodiment of the present invention the characteristic indicators for a condition comprise one or more of the following indicators: total length of writing on paper, stroke width, stroke height, stroke length, speed of writing, acceleration of writing, length of time the writing instrument stays in the air, length of time the writing instrument stays in on paper, the trajectory of the writing instrument in the air, pen tilt, azimuth, coefficient of variance, peak velocity, the pressure applied while writing or any combination thereof.

In a further preferred embodiment of the present invention, analyzing the values of the recorded indicators comprises correlation between two or more recorded indicators.

In yet another preferred embodiment of the present invention the list of characteristic indicators for a condition is established by giving a set of handwriting tasks to a first group known to have said condition and giving the same set of handwriting tasks to a control group known not to have said condition, and analyzing the recorded indicators of the two groups in order to establish which measured indicators characterize the first group.

In yet further preferred embodiment of the present invention the list of characteristic indicators for a condition is established by giving a set of handwriting tasks to a person when the person is known to have said condition and then giving the same set of handwriting tasks to the same person when the person is known not to have said condition, and analyzing the recorded indicators of the two sets of handwriting tasks in order to establish which measured indicators characterize the person when having said condition.

In yet another preferred embodiment of the present invention the handwriting tasks involves writing letters, numbers, drawings or any combination thereof.

In yet further preferred embodiment of the present invention, the method further comprises the step of validating the handwriting analysis results with additional standardized tools.

In yet another preferred embodiment of the present invention the handwriting data is collected by a digitizing tablet.

In yet further preferred embodiment of the present invention the handwriting tasks are functional, everyday tasks including but not limited to: writing own name, writing the alphabet sequence from memory and copying a text.

In yet another preferred embodiment of the present invention the diagnosis method further comprises the step of providing the person with a self-evaluation questionnaire and integrating the responses to the questionnaire in the diagnosis of the condition. If the person at hand is a child or a person unable to fill the questionnaire on his own, it is possible to give the questionnaire to a parent, guardian or any another adult capable of providing meaningful answers regarding that person.

The self-evaluation questionnaire comprises questions regarding possible implications on every day performance and participation in personal, social and professional activities.

In yet another preferred embodiment of the present invention the recording of the handwriting tasks of the person is done using multidimensional computerized systems.

In yet another preferred embodiment of the present invention drawing of complex figures or drawing of figures such as a clock, person, tree, home is used to diagnose a condition. The present invention includes several inventive applications, including, by way of non-limiting examples:
Graphological analysis can be enhanced by creating an objective set of measurable indicators, thus a computer will be able to both record the hand writing and also provide an analysis based on the recorded indicators. In addition, the analysis can be more precise as the computer measures each indicator precisely, while validating the graphologist's written products scoring based on established graphology-defined characteristics. For example, in addition to a human judgment about letters directions/angles or their locations on line, the application of the invention is able to tell in what angle the letter leans, and how much precisely does it extend above or below a line.
Evaluating the effect of a medicine or drug (or any other treatment) on the functional capabilities of a person. For example, did giving Ritalin improve a child with ADHD functional capabilities? Analyzing the child's handwriting after taking the drug can reveal if his concentration is improving or not.
Lie detection—after analyzing the writing of a person writing a knowingly true text and then writing a knowingly false text, the system of the invention can analyze a new text from the same person and determine the likelihood that the text is true or false by comparing it to the previously analyzed true and false texts.
Expert vs. novice—Employees operating machines or performing complex tasks must do many of them automatically in order to be experts (i.e. perform well). For example, an expert driver performs many tasks automatically (changing gear, checking the dashboard display, braking, steering) while the novice has to perform each task in a controlled manner. Certain types of cognitive tasks may become automatic with extensive practice. What is initially effortful and resource-consuming becomes, with practice, automatic and relatively resource independent. The diagnosis system of the invention can check levels of automaticity during performance of tasks, and thus indicate when expertise is reached, and which tasks require more practice in order to achieve it. For example, subjects can write about the condition of a vehicle while looking at slides of the dashboard, and the difference between expert and novice driving can then be checked with the detectable indicators of the invention.
Determining personality or expertise traits. For example, a company wishing to recruit a person with one or more desired conditions (i.e. organized, innovative, creative etc.) can use the system of the invention to identify the characteristic indicators in the handwriting for people known to have the desired condition, and then test the handwriting of candidates to see their match with the desired condition or conditions.
Education field—The diagnosis system of the invention can collect real-time information about automatic mental processes during performance of tasks, showing in detail the performance of students during training, and thus compare the efficacy of educational programs. For example, the system can compare two mathematical training programs by collating handwriting information during performance of basic skills such as division, multiplication, subtraction etc. The system can also show in detail in which program students mastered mathematical skills, assuming that students who mastered these skills are performing them automatically. Such evaluation can also serve to improve individual student performance by pointing to tasks and subtasks performed in a controlled manner. For example, in long division, students who have mastered the skill automatically identify the remainder, while others calculate the remainder in a more controlled manner. The system can indicate which steps and tasks need further study.
Signature or handwriting authentication—a forger can imitate a signature or handwriting so that the forged signature or handwriting looks visually similar to the original signature or handwriting for a personal looking at both samples. However, if both the original and the forged signature or handwriting were written and analyzed using the system of the invention, then the system would be able to detect the forged signature or handwriting from the original since even if the outcome looks very similar, the way the original person and the forger write are not the same. Looking at the measured indicators would indicate that a different person has written the two samples.

In another aspect, the present invention relates to a diagnosis system based on handwriting analysis, comprising:

(i) a list of characteristic indicators for each dysfunction detectable while performing a set of handwriting tasks;

(ii) a digitizer on which the handwriting is done;

(iii) a recording unit for recording the handwriting tasks of a person according to multiple indicators; and

(iv) an analyzer for analyzing the recorded indicators in order to determine whether the person can be diagnosed with said condition.

### BRIEF DESCRIPTION OF THE DRAWINGS

FIG. 1 shows the conceptual framework adopted by the International Classification of Functioning Disability and Health (ICF), taken from the International Classification of Functioning, Disability and Health (World Health Organization, 2001, p. 18).

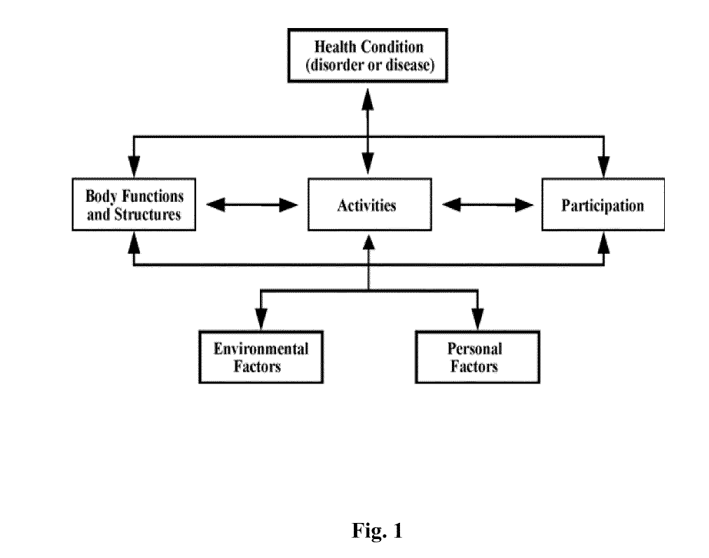

FIGS. 2A-2C show in air measures for a typical subject from each group, a healthy person in FIG. 2A, one with Mild Cognitive Impairments (MCI) in FIG. 2B and a man with Alzheimer disease in FIG. 2C.

FIGS. 3A-3C illustrate a paragraph copying task as performed by a child without ADHD (FIG. 3A); by a child with ADHD on medication (FIG. 3B); and by a child with ADHD, off medication (FIG. 3C). Heavy lines show when the pen was in contact with the paper; thin lines show when it was in the air.

FIG. 4A—shows a target sentence as it appears on the computer screen. That sentence is then copied by both a dysgraphic writer and a proficient writer. The left panels in FIG. 4B show the handwriting of the dysgraphic writer, while the right panels in FIG. 4B show the handwriting of the proficient writer.

FIG. 5A shows a target word as shown on the computer screen (one of the words from the sentence shown in FIG. 4A in this example). FIG. 5B shows the word as written by a proficient writer while FIG. 5C shows the word as written by a dysgraphic writer.

FIG. 6 shows An example of true (top) and false (bottom) writing paragraphs by the same writer.

FIG. 7 shows an illustration of the pen's azimuth measure.

FIG. 8 shows illustration of the segment's spatial measures.

### DETAILED DESCRIPTION OF THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

Standardised evaluation tools for several aspects of performance and participation were already developed by the author (see rosenblum 2006; 2008) and were analysed in relation to handwriting process measures (for examples see: Rosenblum, & Livneh-Zirinsky, 2008; Rosenblum, Weiss, & Parush, 2004). The computerized system developed by the author for gathering handwriting process data indicated that handwriting kinematics analysis is a promising tool for performance-based activity evaluation.

The diagnosis system of the invention comprises a handwriting data collection and analysis software and accompanying tools for performance and participation evaluation which suit every human condition such as but not limited to a dysfunction, pathology or other human situation (early detection in developmental context, aging, lie detection) characteristics and needs.

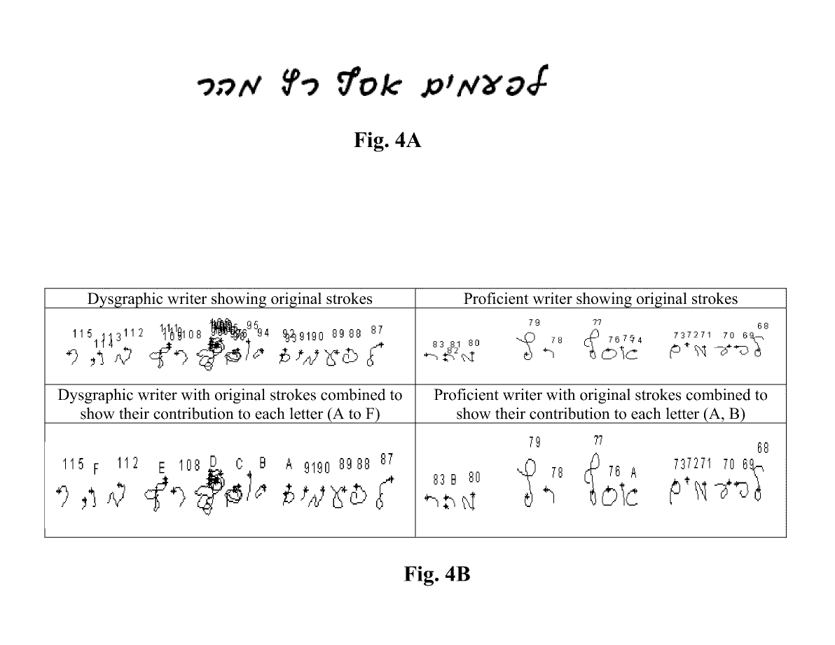

The handwriting data analysis part is built upon a combination of methods from multidisciplinary knowledge means such as mathematics, pattern recognition, signal processing, biology, and graphology. For example, FIGS. 4A-4B represent the information about the handwriting process of a typically developed child in comparison to a child with dysgraphia. The visual presentation is based on writing strokes analysis coming from the signal processing field.

Both the software and additional analysis tools were developed from a point of a deep understanding and knowledge about human abilities and the meaning of the requirements imposed to a person while performing everyday tasks in life. The uniqueness of the invention is in defining the task, the required analysis measures and the combined evaluation tool required in order to accomplish each of the tested task.

Current handwriting analysis software applications suffer from several disadvantages:

1. None of the current handwriting analysis software applications were developed by a professional who deeply understands human being performance as manifested both in the tasks given to the patient to perform and in the analysis options that were developed for the data.

As a result, the existing systems are limited with regards to the amount of written data (task length) because the developers didn't think about functional everyday tasks required for normal human everyday performance, such as copying a long paragraph or filling a check.

2. None of the current handwriting analysis software applications suggested combining other evaluation tools for completing the individual's performance characterization.

3. Mostly, existing software applications are language dependent.

4. There has been a rich knowledge for years that handwriting may manifest one's abilities and several graphological methods are being implemented around the world. However, these methods are based on subjective impression of the evaluator and not on an objective data. These data may be received in an objective, precise way through the ComPET.

5. Although there are a lot of studies about handwriting in the area of pattern recognition and signal processing, the existing knowledge is not implemented to the clinical area; their main purpose is to develop the ability of handwriting recognition by computers and an identification of a person through his handwriting. These methods were not implemented for clinical use in order to diagnose a condition such as a dysfunction, pathology, character trait, skill, expertise, mental condition, truth writing etc.

Furthermore, there are no applications of biological or physical methods (i.e. microscopically analysis or time series analysis) for handwriting in order to get more data about one's functional/dysfunctional performance.

Hence the system of the invention comprises handwriting data collection and analysis software (ComPET), a digitizer on which the handwriting is done, and evaluation and analysis tools.

For each condition, if available, a standardized tool which is in common use for the diagnosis of that conditions is used. For example, in order to establish the medical diagnosis MMSE is used for diagnosing for Alzheimer, GDS for depression, M-ABC for children with DCD etc.

The handwriting tasks that are developed are sensitive to the specific condition in question.

Analysis methods are implemented to the data combining multidisciplinary exiting knowledge that was not implemented yet for the clinical needs.

A companion evaluation tolls for a self report by the person or his caregivers about every day performance is developed according to the unique characters of each condition and is to be filled in by the patient.

### Building A New Study

Following is an illustrative but non limiting example of how to analyze a condition:

1. A comprehensive data file is built from available studies in the field. The data file includes over 1500 subjects. Around half of the subjects exhibit normal behavior and handwriting and the other half exhibit various conditions.

Statistical methods are implemented to the data file in order to find a common principle of dis-automaticity in handwriting that characterizes all the conditions.

2. New handwriting data is collected. All subjects perform the same handwriting tasks, such as writing one owns name, the alphabet sequence, paragraph copying and additional tasks, according to the unique characteristics of the specific condition being analyzed.

The population groups are be studied also in different countries in order to get data about the same populations also in foreign languages and try to find whether the measures that will be found as best differentiators will be non-language dependent measures.

3. New data analysis methods are developed in a dynamic process within an interdisciplinary team as described above.

4. A survey of available software/hardware that may enrich the data collection phase is carried out and possible enhancements can be combined with the ComPET. For example, systems (software, hardware and supplementary tools) for measuring reaction time, pressure implemented to the fingers while writing and eye tracking.

The developmental sequence is analysed based on the available data in the languages of the population. The new collected data enables to check whether same patterns are similar for people with certain conditions, although they wrote in different language.

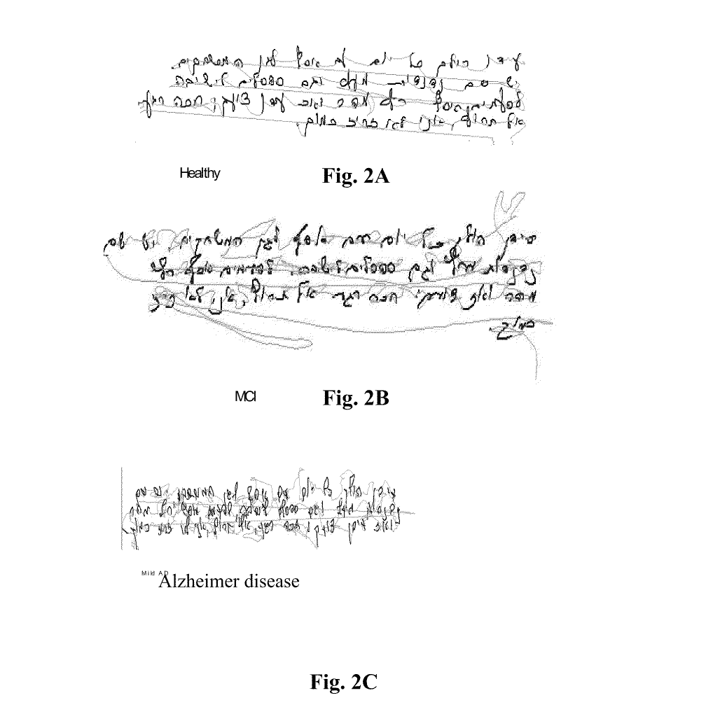

5. After visualization methods (such as those presented on FIGS. 2-4) are developed, people who participated in the new data collection phase are asked to see their handwriting measures and react and give feedback regarding the results. Their input may leads for development of further data analysis and/or visualization techniques.

6. Based on all the new and old data, a model of handwriting performance is then developed. Based on the new data a model of possible relationships between the temporal, spatial and pressure handwriting data and the results of the self report performance and participation questionnaires is built.

### EXAMPLE 1

### Medical Diagnosis Methodology

Available handwriting data is analysed with a multidisciplinary team. Data from several populations is gathered in the following order:

1. A background literature about the condition and its characteristics is performed, in this case a medical condition.

2. Considering possible implications on every day performance and participation, an interview guide is prepared which includes questions regarding those aspects.

3. The study population is medically diagnosed by a medical doctor preferably aided by a standardised tool commonly used in order to diagnose this medical condition.

4. After getting an ethical approval and informed constant, an interview of the patients about their everyday performance and participation characteristics is performed.

5. Interview content is analyzed. Based on the results it is determined whether a performance and participation questionnaire which is sensitive to this population's characteristics is available or whether a new tool needs to be developed.

6. Specific handwriting tasks are included, based on the population's specific needs.

7. Based on the available handwriting data and the new data, sophisticated methods for the handwriting data analysis is developed in a multidisciplinary team.

### Participants

Handwriting data has been gathered in the past in multiple languages from children and adults in various age groups having various conditions such as dysfunctions or pathologies. The available publications by the author represent only part of the data that is available in the field.

The purpose of the invention is to develop deeper analysis methods for the available data but also to continue and collect data for other purposes, for example, the populations described in Table 1 below.

The rational for choosing these populations is that there are already clues about handwriting deficiencies among them but the available studies are not sufficient in order to better understand the process and the relationships between the process measures and their participation abilities.

TABLE 1
Experience Population
Population

60 people diagnosed with Parkinson disease and 60 controls
60 pre-schools children suspected by their teacher as clumsy
(DCD) and or having perceptual motor deficits and 60 controls
60 students and adults ages 20-40 who are suspected for DCD
and 60 controls
30 people diagnosed as Major Depression and 30 controls
30 people diagnosed with Schizophrenia and 30 controls
30 children with Neurophibrimatiosis and controls

### Instruments

Computerized Penmanship Evaluation Tool (ComPET, previously referred to as POET; Rosenblum, Parush, & Weiss, 2003). This standardized and validated handwriting assessment utilizes a digitizing tablet and on-line data collection and analysis software. It was developed for the purpose of collecting objective measures of the handwriting process (see Rosenblum et al., 2003 for more details). The ComPET system is non-language dependent and analyzes every writing stroke.

The data collection part is simple to operate and use, and the data analysis can be done by the researcher.

FIGS. 2A-2C show in air measures for a typical subject from different groups. FIG. 2A shows the writing pattern of a healthy person with an in-air time of 20 seconds while writing the paragraph. FIG. 2B shows the writing pattern of a person with Mild Cognitive Impairments (MCI), and who's an in-air time for writing the same paragraph was 57.76 seconds. FIG. 2C shows the writing pattern of a man with Alzheimer disease, and who's an in-air time for writing the same paragraph was 84.33 seconds.

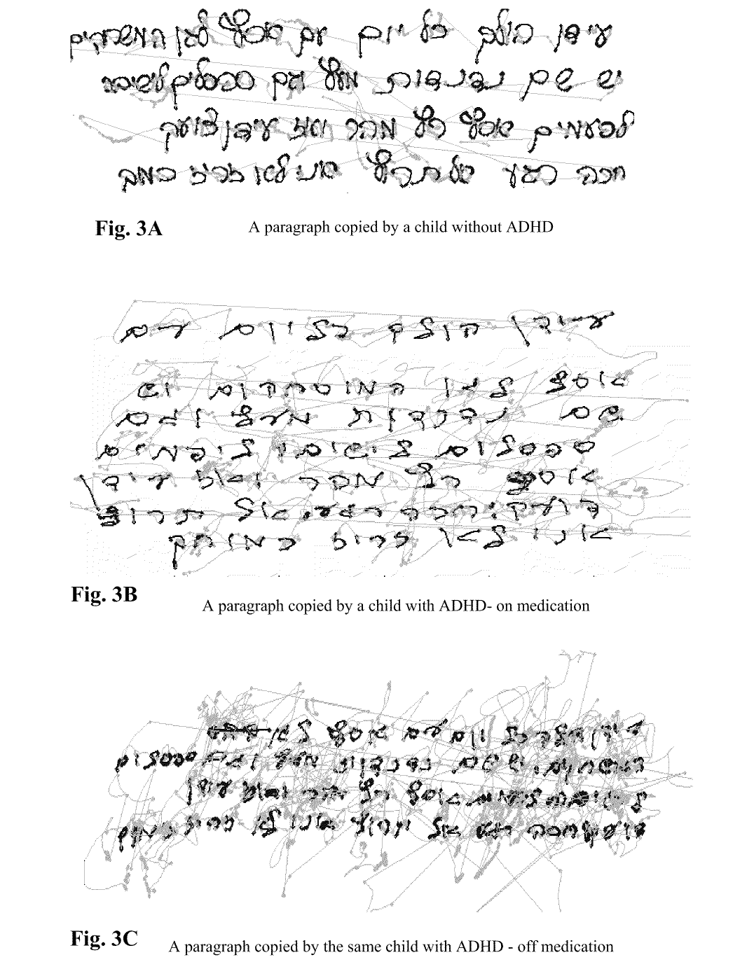

FIGS. 3A-3C illustrate a paragraph copying task as performed by a child without ADHD (FIG. 3A); by a child with ADHD on medication (FIG. 3B); and by a child with ADHD, off medication (FIG. 3C). Heavy lines show when the pen was in contact with the paper; thin lines show when it was in the air.

The tasks to be performed are functional everyday tasks. In most populations it includes writing owns name, writing the alphabet sequence from memory and paragraph copying. These tasks were chosen from an ecological point of view (i.e. tools that reflect real life) as very familiar and common tasks. Further tasks are included according to the specific population characters and needs. The tasks were performed on A4-sized lined paper affixed to the surface of a WACOM Intuos II x-y digitizing tablet (404×306×10 mm), using a wireless electronic pen with a pressure-sensitive tip (Model GP-110). This pen is similar in size and weight to regular pens commonly used by children and thus does not require a change in grip that might affect their writing performance.

FIGS. 4A-4B show a four word sentence as it appears on the computer screen (FIG. 4A), and (FIG. 4B) as written by a representative dysgraphic writer (left panels) and by a proficient writer (right panels).

FIG. 4B illustrates the ability of the routines to automatically divide up the text into individual segments. Each segment is designated with a number which shows the order in which it was written. It is therefore possible to track the sequence of writing.

In the sentence written by the proficient writer, just two segment combinations were necessary, i.e., the combination of segments 74 and 75 which has been labeled as A in FIG. 4B (lower right panel) and the combination of segments 81 and 82 which has been labeled as B in the same panel. Since both of these characters are normally constructed from two components, these combinations are entirely expected. In contrast, when comparing the upper and lower left panels, one can notice that six segment combinations were required for the sentence written by the writer with dysgraphia (labels A to F in the lower left panel of FIG. 4B). One letter in this same panel (the letter “Pey″” in its complex form indicating word termination) was combined from seven segments (segments 99-107 in the upper left panel, labeled as D in the lower left panel). The same letter was written in one segment (77) by the proficient writer (upper and lower right panels). Note that this letter is normally written as a single unit, and has been done so by the proficient writer as demonstrated by the lack of combinations in the lower right panel of FIG. 4B.

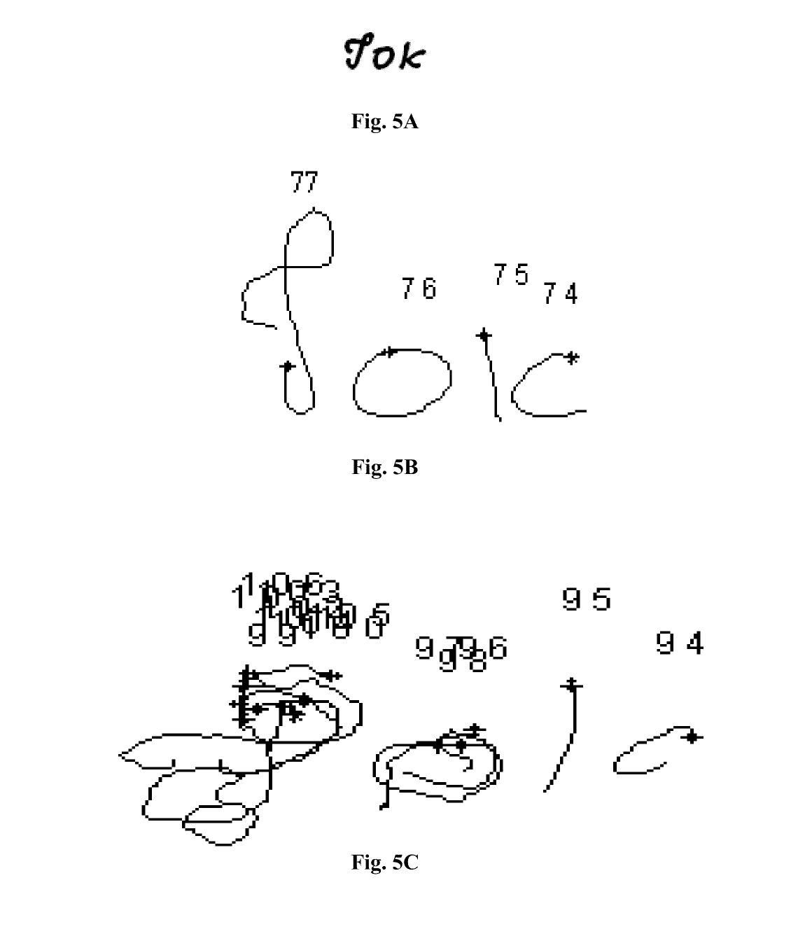

FIG. 5A shows a target word as shown on the computer screen (one of the words from the sentence shown in FIG. 4A in this example). FIG. 5B shows the word as written by a proficient writer requiring just four segments, the minimum possible number. In contrast, FIG. 5C shows the word as written by a dysgraphic writer requiring 14 segments.

Displacement, pressure, and pen tip angle were sampled at 100 Hz via a 1300 MHz Pentium (R) M laptop computer. The primary outcome measures were comprised of temporal, spatial, and pressure measures for each writing stroke, as well as performance over the entire paragraph. The temporal measures included on-paper time and in-air time (i.e., the time during writing performance in which the pen is not in contact with the writing surface) (Werner, Rosenblum, Bar-On, Heinik, & Korczyn, 2006). In previous studies, we found that in-air time may supply information about the perceptual aspect of the motor act (e.g., Werner et al., 2006);

hence, we decided to separate the temporal measure into on-paper time and in-air time. The spatial measure used was the mean stroke height and width for each task. In addition, the ComPET computes the mean pressure applied to the paper, as measured in non-scaled units from zero to 1024, as well as the mean pen tilt in the range of 0°-90° (i.e., the angle between the pen and its projection on the tablet).

### Developing Data Analysis And Visualization Techniques

A multidisciplinary team is formed to first recognize any data analysis and visualization methods that are well known for handwriting but still haven't been used for clinical needs.

Based on previous experience, a dialog takes place between the principal investigator and the experts based on the handwriting data files, what is required and what is indeed existent in their respective fields.

Parallel, new algorithms and other methods are developed based on the identified requirements of each condition to be diagnosed.

The team may include experts in one or more of the following areas:

1. Forensic handwriting analysis, including expertise in pattern recognition and/or signal/image processing

2. Mathematics

3. Statistics

4. Computer applications

5. Hardware, electronics

6. Physics

Segmentation, in forensic terms involves the process of script examination, most usually done for separate letters—Forensic handwriting analysis (Cohen)

Their main issue is to deal with signature identification in legal and financial contexts (Koppenhaver, 2002, 2007).

In the context of legal and illegal activities, there applications and articles for the police force, lawyers, forensic scientists and private detectives about handwriting specimen's analysis (e.g., Morris, 2000) as well as about the effect of legal and illegal drugs on handwriting (Wellingham-Jones, 1991).

In the computer science field, using neural network and algorithms can be helpful for handwriting characters recognition (e.g., Oh & Suen, 2001; Plamondon & Srihari, 2000). The pattern analysis may be implemented to the writing product which pertains to scanned images (offline recognition) or while the handwriting is performed (online recognition) (Plamondon & Srihari, 2000).

### EXAMPLE 2

### Lie Detection

In this study we compared the handwriting of the same individuals when asked to write truthful and deceptive sentences. Our research hypothesis is that differences will be found between writing of truthful sentences and writing of false sentences in pressure, temporal (stroke duration on paper and in air) and spatial measures (strokes path length, height and width) obtained by the computerized system. Based on the finding of the clinical studies we predict that in deceptive writing, the mean and standard deviations of handwriting measures of each participant will be varied. Thus while writing deceptive sentences, higher pressure will be implemented, longer duration time per stroke (on paper and in air) will be required, and letter strokes will be larger in comparison to truthful writing.

### Methodology

### Participants

Participants were 34 healthy students, including 25 females and 9 males, aged 20-35 (mean age 25.51, SD¼3.41), who were recruited at the University of Haifa in northern Israel. Seventy per cent of the participants were born in Israel, while 27% were born in the former Soviet Union and 3% in Europe. The majority (85%) of the participants had right-hand dominance, and 15% were left-handed.

The criteria for inclusion were: residence in Israel for at least 20 years; normal or corrected to normal vision and hearing ability; at least 13 years of education; and a minimum of three sentences in Hebrew written at least three times a week. Anyone suffering from any form of neurological/emotional or physical disease was not eligible to participate in the study.

### Instruments

The socio-demographic questionnaire included gender, age and number of years of education.

Digitizing tablet and online data collection and analysis software: The objective spatial, temporal and pressure measures were provided by the Computerized Penmanship Evaluation Tool (ComPET).

All writing tasks are performed on A4 lined paper affixed to the surface of a WACOM Intuos 2 (model GD 0912-12×18) x-y digitizing tablet, using a wireless electronic pen with a pressure-sensitive tip (Model GP-110). Displacement, pressure and pen-tip angle are sampled at 100 Hz by means of a 1300 MHz Pentium (R) Mlaptop computer. The digitizer provides accurate temporal measures throughout the writing, both when the pen is touching the tablet (On-paper time) and when it is raised (In-air time). It also provides accurate spatial measures when the pen is touching the tablet and/or when it is lifted above the digitizer up to 6 mm. Beyond 6 mm, the spatial measurement is not reliable.

The ComPET analysis results in several measures:

Pressure measure—the mean pressure implemented towards the writing surface for the entire task measured in non-scaled units from 0-1024. Whereas the other measures are related to writing strokes and not to whole letters or the whole task, this measure is not specific for a single stroke but for the entire task. Stroke refers to the curve created by the movement of the pen-tip on the paper, which is represented on the X, Y coordinate system. That is, the computerized analysis does not recognize letters but points while writing, when the pen is in contact with the paper and those in which the pen leaves the paper. It is important to note that there is variability between and within writers. That is, some will write the same letter with one continuous stroke while others will write it with several strokes, and the same individual may write a letter in one stroke once and in several strokes in other words within the same sentence. Therefore, in alignment with the clinical technique of analyzing handwriting behavior, we chose to measure aggregated measures of the entire task. The mean as well as the standard deviation of each measure was examined for each participant in order to follow the intra-individual variability across different measures:

1. Temporal measures: Stroke duration in air (while the pen is not in contact with the writing surface) and on paper, both measures reported in seconds.

2. Spatial measures:

2.1 Stroke path length in millimeters, which measures the total path length from the starting point to the finishing point for each written stroke.

2.2 Stroke height (on the Y-axis), which measures the direct distance from the lower point of the stroke to the highest point in millimeters.

2.3 Stroke width (on the X-axis), which measures the direct distance from the left side of the stroke to the right side in millimeters.

Number of peak velocities per stroke: A measure for handwriting movement regularity, with the assumption being that the more peaks there are in one stroke, the less regular the movement will be.

Based on previous handwriting analysis, the coefficient of variance (the standard deviation divided by the mean) for the stroke duration, path length, height and width was analyzed as a measure of the consistency of handwriting performance.

Several studies have indicated the ComPET's validity for differentiating between children with and without dysgraphia (handwriting difficulties); and Multiple Sclerosis. The system has also been shown to differentiate between age groups.

### Procedure

Signed informed consent was obtained from the participants following approval by the Ethical Committee of the University of Haifa. Advertisements at the University were used to recruit students to participate in the study. Based on Johnson and his colleagues (Johnson, Foley, Suengas, & Raye, 1988), the participants were asked to write two short paragraphs in sequence describing autobiographical events and memories, one about a true event and the other a false description of the same event. The students were requested to write the true and false paragraphs in Hebrew (about five lines) on a paper that was affixed to the digitizing tablet. The order of the true and false events was varied, with half of the participants writing the description of the true event first and the other half writing the description of the false event first.

### Data Analysis

Descriptive statistics of the dependent variables were tabulated and examined. The number of strokes for the truth and false paragraphs were compared by paired sample t-test. Following the finding that there were significant differences between the groups for the number of strokes, a measure of the difference between number of strokes at the truth task and number of strokes at the false task was computed (d-stroke).

Two MANOVAs were done for each of the following three types of measures, one to the mean values and the other MANOVA to the standard deviation of the values.

1. Pressure implemented towards the writing surface.

2. Temporal measures (stroke's duration in air and on paper).

3. Spatial measures (stroke's path length, width and height).

Further MANOVA was done for the coefficient of variance of the measures (stroke duration, path length, height and width) and the peak velocities measure.

It is important to note that all the data collection is performed automatically by the ComPET data collection part, in real time while the subject is writing. This data, obtained as a text file, is objective and exact data with physical nature (length, time and pressure measures). The raw data is then aggregated to a final measure with the ComPET data analysis part based on MATLAB with no subjective interpretation by the researcher.

### Results

T-test analysis indicated significant differences between the truth writing paragraphs and the false paragraphs for the number of written strokes (truth M=420.06, SD=129.71; False: M=350.00, SD=97.33, t(33)=3.50, p=0.001).

TABLE 2
Comparison of the pressure's means and standard
deviations for true and false writing

| Pressure | True n = 34 | False n = 34 | | | |
|---|---|---|---|---|---|
| measures | Mean(SD) | Mean(SD) | F (1.33) | p | ES ² |
| Mean | 879.52 (80.42) | 893.52 (72.69) | 5.89 | .021 | .152 |
| pressure | | | | | |
| Standard | 162.97 (17.89) | 160.47 (17.95) | 2.15 | N.S | .061 |
| deviation | | | | | |
| pressure | | | | | |

Hence a measure of the difference was computed (number of strokes in the truth writing—number of strokes in the false writing) for each participant (d-stroke) and was held as constant while conducting the following MANOVA's with repeated measures.

1. The MANOVA analysis's indicated that the mean pressure implemented towards the writing surface in the false writing was significantly higher in comparison to that implemented in the truth writing (False: M=893.52, SD=72.69; truth M=879.52, SD=80.42, F(1.33)=5.89, p=0.021, ES²=0.15). No significant differences were found for the pressures standard deviation measure (see Table 2).

2. The results of the MANOVA with repeated measures done for the means and standard deviations of the temporal measures indicated no significant differences between truth and false writing (F(2.31)=0.246, p=0.78, ²=0.016) The means and standard deviations are presented in Table 3.

TABLE 3
Comparison of the temporal measures (stroke duration on paper and
in air)-means and standard deviations for true and false writing

| | True n = 34 | False n = 34 | | |
|---|---|---|---|---|
| Temporal measures | Mean (SD) | Mean (SD) | F (2.31) | p |
| Mean stroke duration on | 0.150 (0.026) | 0.153 (0.026) | .153 (.026) | N.S |
| paper | | | | |
| Mean stroke duration in air | 0.242 (0.112) | 0.244 (0.076) | .246 | N.S |
| Stroke duration on paper | 0.092 (0.025) | 0.091 (0.016) | .073 | N.S |
| Standard deviation | | | | |
| Stroke duration in air | 0.582 (0.426) | 0.544 (0.318) | .234 | N.S |
| Standard deviation | | | | |

Table 4 shows the results for the means and standard deviations of the spatial measures.

The MANOVA with repeated measures done for the means of the spatial measures indicated significant differences between truth and false writing (F(3.30)=3.39, p=0.031, ES²=0.253). Post hoc ANOVA indicated that in false writing, the strokes were significantly longer and higher (see Table 4).

Although no significant differences were found for the MANOVA of the standard deviations of the spatial measures (F(3.30)=2.13, p=0.117, ES²=0.176), post hoc ANOVA indicated significant difference between the true and false writing for the standard deviation of stroke height (F(1.32)=4.73, p=0.033, ES²=0.134).

The MANOVA with repeated measures conducted for the measure's Coefficient of variance (stroke duration, path length, height and width) and peak velocity indicated no significant differences between truth and false writing (F(5.28)=0.64, p=0.67, ²=0.103).

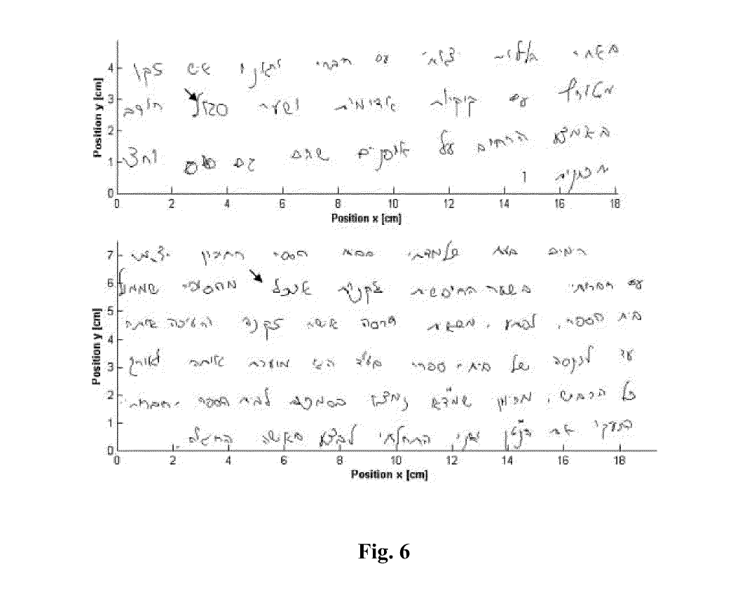

An example of the handwriting paragraphs of one participant is presented in FIG. 6 in order to illustrate the differences between the true (top) and false (bottom) paragraphs.

The differences are also presented for one specific stroke, the letter L (“lamed”) in Hebrew, which was chosen in both paragraphs in the same location (two examples in which the writer wrote the letter in one stroke and not in several strokes). The analysis software points to the number of the stroke and the designated letter in both paragraphs as being the 70th stroke. The sizes of the 70th letter in each paragraph (sign by arrow) appear in Table 4.

TABLE 4
Comparison of the spatial measures (stroke's path length, width
and height)- means and standard deviations for true and false writing

| | True n = 34 | False n = 34 | | | |
|---|---|---|---|---|---|
| Spatial measures | Mean (SD) | Mean (SD) | F (3.30) | p | ES ² |
| Mean stroke length | 0.554 (0.141) | 0.590 (0.147) | .598 | .020 | .158 |
| Mean stroke width | 0.201 (0.050) | 0.212 (0.048) | 2.27 | N.S | .066 |
| Mean stroke height | 0.262 (0.076) | 0.278 (0.077) | 10.33 | .003 | .244 |
| stroke length | 0.411 (0.079) | 0.429 (0.097) | .569 | N.S | .017 |
| Standard deviation | | | | | |
| stroke width- | 0.201 (0.050) | 0.145 (0.031) | .206 | N.S | .006 |
| Standard deviation | | | | | |
| stroke height | 0.163 (0.034) | 0.172 (0.038) | 4.93 | .033 | .134 |
| Standard deviation | | | | | |

Table 5 presents the length and height measures for that specific stroke made by that one participant.

TABLE 5
A visual presentation of the 70th stroke of one participant, as appears
in true and false writing, and the stroke's length and height

Variables

| The letter appearance | True | False |
|---|---|---|
| Mean stroke length (mm) | 9.90 | 10.56 |
| Mean stroke height (mm) | 4.75 | 7.40 |

In order to further illustrate the differences, the measures of two representative participants when writing true and false paragraphs are presented in Table 6.

TABLE 5
Examples of two writers' (1 and 2) mean stroke length/height/width,
standard deviation values as appears in their true and false writing

| | Participants Variables | | | |
|---|---|---|---|---|
| | 1 | | 2 | |
| | True | False | True | False |
| Mean stroke length (mm) | 0.51 | 0.59 | 0.60 | 0.65 |
| Mean stroke height (mm) | 0.23 | 0.28 | 0.26 | 0.27 |
| Mean stroke width (mm) | 0.18 | 0.21 | 0.23 | 0.25 |
| Standard deviation of stroke length (mm) | 0.36 | 0.38 | 0.44 | 0.56 |
| Standard deviation of stroke height (mm) | 0.15 | 0.17 | 0.18 | 0.23 |
| Standard deviation of stroke width (mm) | 0.12 | 0.13 | 0.15 | 0.18 |

### Discussion

In this preliminary study, we applied the ComPET for purposes of detecting deception. It was assumed that greater mental complexity would be evident in the handwriting of participants when describing false autobiographical events or memories as opposed to true events. Temporal and spatial measures were derived for each writing stroke, as well as a pressure measure and number of peak velocities for the entire task, in order to enable a comparison of the performance between writing true and false statements. Results show that in the false writing condition, the mean pressure, stroke length and height were significantly higher than in the true writing condition. Furthermore, the standard deviations of stroke heights were significantly higher in the false condition than in the true condition.

Our results support the automatic and controlled information processing model. It seems that in a task with higher mental load, such as writing a lie, the automatic process involved in normal handwriting is replaced with a more controlled process, which is sensitive to task difficulty and thereby limits dual-task performance.

Furthermore, it seems that by virtue of concentrating more on the deceptive cognitive task, subjects are more limited in their movements in order to save cognitive resources.

Their limitations in writing movements manifested in higher stroke length and height (and standard deviation), which together indicate less regularity.

In relation to the temporal measure and unlike previous studies, we measured stroke duration and not reaction time in the present study.

Based on the lack of significant differences for both ‘on paper’ and ‘in air time’ in the deception writing, it seems that these measures are not sensitive enough to deception writing, despite the fact that they have previously been found sensitive to cognitive deficits in pathologies such as Alzheimer disease. Another option may be that it depends on the kind of task given to the subject, a point which could be elaborated in future studies. Along the same lines, the fact that no significant differences were found for the coefficient of variance of the measures as well as for peak velocity may indicate that in the case of lie detection, the focus needs to be on the differences in the spatial characters of the strokes, that seems to be alerted as a result of the cognitive load.

These results suggest that a documentation of handwriting process measures with a computerized system such as ComPET while focusing on amount of automatization/regularity may be used as another tool for lie detection. Such a tool may have advantages over other lie detecting methods in that it is not intrusive and is user friendly. It can improve the accuracy of other lie detectors by offering additional measures to existing ones in order to reduce errors of interpretation. Furthermore, though other methods are useful in detecting lies during verbal communication, the ComPET is the only measure that we know of which can be used to detect lies in written communication.

The ComPET is an easy to use system that generates objective data automatically, which cannot be obtained manually by observing handwriting behavior or by analyzing written text. Measures such as the standard deviation of stroke height of each participant or pressure applied are unique measures received easily and in an objective way. Furthermore, the writer is not aware of the kind of data being measured and, even if aware, measures such as writing pressure, stroke height, width or standard deviation of stroke height cannot be actively controlled in a consistent way. The analysis done to strokes and not to letters enables implementation of this technique to writing in various languages. Overall, we find this technique useful for researchers and practitioners studying deception.

Although one short task was used for this preliminary test, future studies could employ a variety of tasks that may be even more effective in detecting deception. For example, these tasks could use more complex deception scenarios and measure the ground truth base line more systematically.

### Example 3

### Computerized Kinematic Analysis of the Clock Drawing Task In Elderly People With Mild Major Depressive Disorder

Clock drawing test (CDT) is a term used to collectively describe a group of different approaches designed for cognitive assessment central to which is a request to draw/identify a clock face and/or its components, subsequently evaluated and scored according to pre-determined criteria or concepts. Clock drawing may, in addition, be incorporated to form part of other cognitive instruments or be used in combination with other cognitive tests. Given its brief administration time, simplicity, confirmed validity to screen for dementia, and its tapping into a series of cognitive domains, with special reference to executive dysfunction, that may be impaired early in dementia, CDT is one of the most widely used cognitive tests. Among the many CDT protocols introduced thus far none has been found consistently, under every circumstance and for every purpose superior to the others to detect cognitive impairment. While the influence of education, language and culture on clock drawing performance has been largely acknowledged, there is a dearth of knowledge regarding the presumed impact of depressive disorders on CDT.

Clock drawing, considered a drawing task in neuropsychological assessment, was generally analyzed only after the patient had completed the task. Yet there is much to be gained from an analysis of the manner in which the drawing was produced. For example, the features of drawing movements involved in clock drawing such as starting position or direction of movements, the so called “process” approach, which emphasizes the value of examining the qualitative aspects of clock drawing might increase our understanding of brain function. However, only a few of the clock drawing methods have incorporated this concept into their scoring protocols and data concerning specifically the role played by this observable aspect are still lacking in cognitively impaired as well as in depressed elderly persons.

The aims of this study were: a. to examine kinematically the clock drawing task in elderly patients with mild Major Depressive Disorder (MDD), as compared with healthy controls; b. to assess the relative importance of kinematic measures for the differentiation of the groups; and c. to analyze the associations between the clock drawing computerized measures and the cognitive and depression status of the study group.

### Methodology

### Participants And Clinical Assessment

The study group included a convenience sample of 20 elderly persons with a DSM-IV (Diagnostic and Statistical Manual of Mental Disorders, Fourth Edition, APA 1994 ) diagnosis of mild MDD, recruited from psychogeriatric clinic referrals following a comprehensive multi-disciplinary assessment (including assessments of a geriatric psychiatrist, a geriatrician, a nurse, a social worker, and routine laboratory tests). Additionally, a control group of 20 healthy volunteers who were recruited among the MDD participants' relatives and were matched for age, gender, and educational level participated in the study. The control group underwent a similar diagnostic procedure with the exception of the laboratory tests. The study protocol was approved by the local Helsinki Committee. The participants' socio-demographic and clinical characteristics and performance on handwriting tasks are described in “Rosenblum et al, 2009, Handwriting process variables among elderly people with mild Major Depressive Disorder: A preliminary study. Aging, Clinical and Experimental Research” and its content is incorporated herein by reference. Briefly, the diagnosis of mild MDD was a consensus diagnosis established by two geriatric psychiatrists following interview with patients and the use of a checklist of DSM-IV operational criteria for Major Depressive Disorder. The mild severity of MDD was characterized by the presence of only five or six depressive symptoms and only minor impairment in occupational functioning or in usual social activities or relationships with others (American Psychiatric Association (APA), 1994). Emotional and cognitive statuses were quantitatively assessed with the 15-item version of the Geriatric Depression Scale (GDS) and the Hebrew translation of the Mini-Mental State Examination (MMSE).

Aside from the psychiatric diagnosis other inclusion criteria were: age 60 and over, living in the community in Israel for at least 20 years, right-handedness, and normal or corrected to normal vision and hearing ability. Exclusion criteria were: dominant hand tremor, weakness or sensory symptoms or signs, a history of drug or alcohol abuse, a Central Nervous System (CNS) disorder or a psychiatric disorder other than mild MDD. In addition, subjects with an MMSE score lower than 25 were not included in the study. The above procedure and exclusion criteria permitted us to reduce the presumed impact of treatment and medications for other conditions on the clock drawing performance.

Participants with MDD were not statistically different from controls regarding age (mean=75.70±6.61 years vs. 74.55±7.63, respectively), gender (75% females vs. 85%, respectively) and years of education (mean=12.25±2.69 vs. 13.60±3.57, respectively). MDD participants had significantly lower MMSE scores compared with controls (26.12±1.02 vs 27.15±0.81, t₍₃₈₎ 3.50, p=0.001 ), however, understandably, higher scores on the GDS ( 7.70+2.00 vs. 0.80+1.05, t₍₃₈₎ 13.63, p<0.001 ).

### Equipment And Clock Drawing Tasks

COMPET was used to administer the stimuli and to collect and analyze the data. The clock drawing task was performed on A4 lined paper affixed to the surface of a WACOM Intuos model GD 0912-12X18 x-y digitizing tablet (available from WACOM Co. Ltd. of 2-510-1 Toyonodai Otonemachi, Kita Saitama-Gun, Saitama, Japan.), using a wireless electronic inking pen [Model GP- 110]. Displacement, pressure, and pen tip angle were sampled at 100 Hz via a 1300 MHz Pentium (R) M laptop computer. The computerized system enables the collection of spatial, temporal, and pressure data while the subject is drawing. The digitizer gives an accurate temporal measure for the total drawing performance time, both when the pen is touching the tablet and when it is in the air. Regarding the spatial measure, the digitizer gives an accurate measure when the pen is touching the tablet and/or when it is lifted up to 6 mm above the digitizer. Beyond 6 mm, the spatial measurement is not reliable, but the temporal measurement is reliable, hence only the spatial measures of drawing while the pen was in contact with the paper were included in the analysis.

Kinematics measures: Based on previous results, we focused on the following measures:

Measures for the entire task:

1. Mean number of drawn segments on the paper—on paper segment defined as the pen trajectory from a point it touches the paper till the point where the pen is not touching the paper. ‘Touching the paper’ means a pressure level above 50 units.

2. Mean performance time to complete the task, in seconds.

3. Mean pressure implemented towards the writing surface in non-scaled units from 0-1024. This means that the units of measurement for pressure are continuous and ranked but were not gram per area.

4. Mean pen azimuth for the entire task—in the range of 0°-360°. The azimuth is the angle between the “North” line, which is the reference line for 0° (and also) 360°, and the pen's projection on the tablet. The “North” line is a line parallel to the Y axis which starting point is the pen's tip; the angle is measured clockwise.

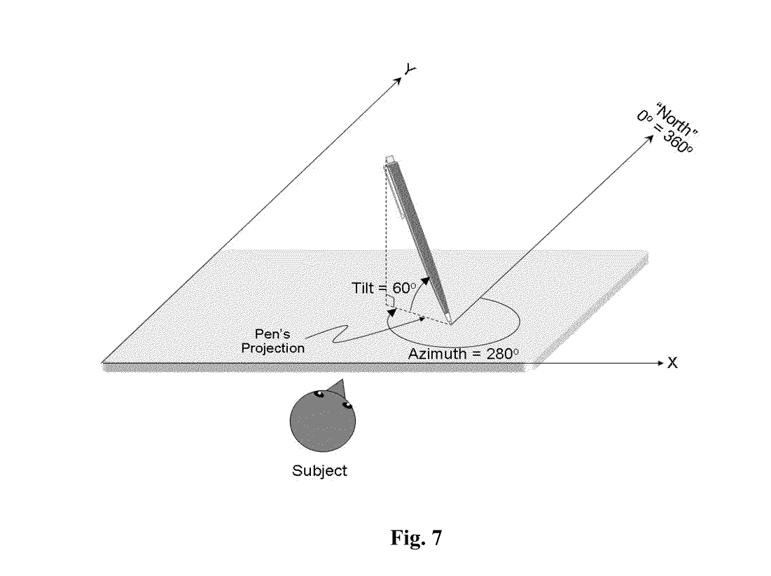

When the pen's azimuth is near and to the right of the “North” line, the azimuth's values will be near 0°, while when the pen is near and to the left of the “North” line, the azimuth's values will be near 360°. Therefore, the azimuth range represents the motion range activating the pen while writing (as shown in FIG. 7).

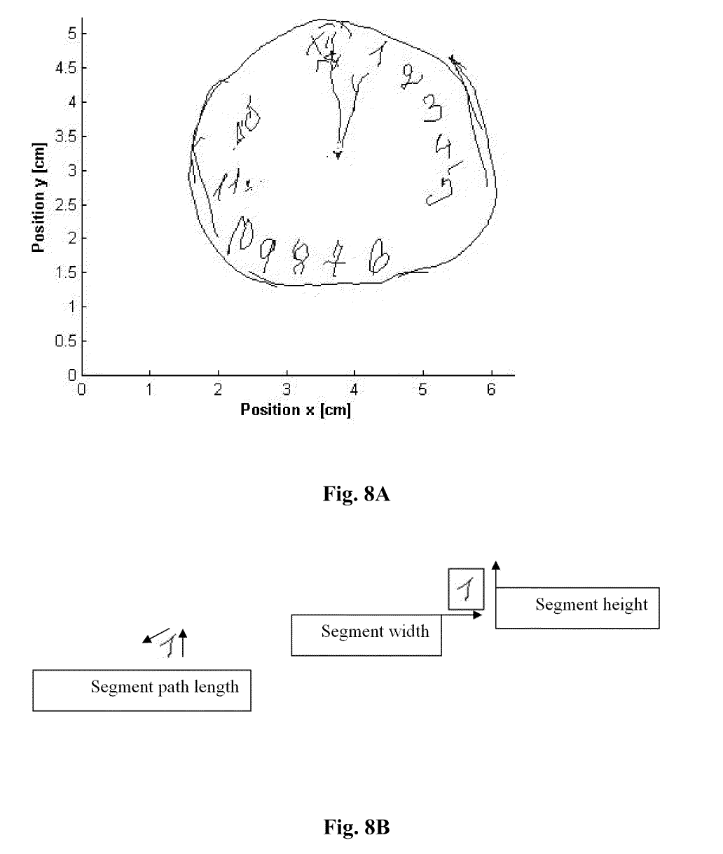

Furthermore, three representative spatial characteristics supplied by ComPET were analyzed per segments written on the paper: (as shown in FIG. 8)

1. The segment's width in centimeter (i.e., the whole segment width ion the x axis)

2. The segment's height in centimeter (i.e., the whole segment height on the Yaxis)

3. The segment's length in centimeter (i.e. the total path length of the pen's trajectory from the point it touches the paper till the point it leaves the paper).

Clock Drawing Task: Participants were presented the A4 lined paper affixed to the surface of the digitizing tablet described above and given the following instruction: “I would like you to draw a clock, put in the numbers and set the time at eleven and ten”. Besides the computerized kinematic analysis conducted, the clock drawings were also scored blindly by one of the investigators according to Freedman et al. (1994) criteria for free-drawn clock. This consists of 15 critical items that constitute a total score of 15 (contour 2 items, numbers 6 items, hands 6 items, center 1 item). Optimal discrimination between well elderly and demented was found using a cutoff of 12 out of 15.

### Statistical Analysis

Descriptive statistics (means, standard deviations) were used to describe the main variables. T-tests were used in order to compare the clock drawing total scores obtained with Freedman's method as well as the kinematic measures of number of on paper segments that were drawn.

Three MANOVA analyses were then used to test for group differences, while controlling for the participants' cognitive status (MMSE scores). The first MANOVA was performed to assess differences in the scores of the clock drawing task (contour, numbers, hands and center), the second MANOVA was performed to assess differences in the computerized process measures of the entire task (total performance time, pressure and azimuth). The third MANOVA was performed to assess differences in the computerized spatial process measures per segment (segment length, width and height). Univariate ANOVA analyses were used to determine the source for the between group differences. Pearson correlations were calculated in order to investigate the associations between participants' clock drawing scores and computerized measures and their depression (GDS) and cognitive (MMSE) scores.

Finally, discriminant analysis was conducted in order to determine which of the clock drawing measures constitutes the best predictors of group membership. Both the clock drawing scores and computerized measures were included in the discriminant function.

### Results

Comparison of clock drawing scores: Table 7 presents the means and standard deviations of the clock drawing total scores and sub-scores in the two diagnostic groups. T-test implemented to the clock drawing total score yielded no significant differences between study group and controls. Mean total scores in both were above the cut-off point of 12 suggested for cognitive impairment (Freedman et al., 1994). Similarly, the MANOVA yielded no statistically significant differences between the groups for each of the subscores (F_{(3.34 )}=1.38, p=0.11).

TABLE 7
Comparison of the clock drawing total scores and subscores

| | Mean (SD) in | Mean (SD) in | |
|---|---|---|---|
| Clock drawing scores | MDD (n = 20) | Controls (n = 20) | p |
| Total score (max 15) | 13.00 (1.85) | 13.45 (1.23) | NS |
| Subscore: contour (max 2) | 1.89 (.46) | 2.00 (.00) | NS |
| Subscore: numbers (max 6) | 5.00 (.88) | 5.30 (.66) | NS |
| Subscore: hands (max 6) | 5.11 (1.15) | 5.15 (.74) | NS |
| Subscore: center (max 1) | 1.00 (.00) | 1.00 (1.00) | NS |

Where: SD—standard deviation; MDD—major depressive disorder; p—significance level; and NS—not significant.

Comparison of clock drawing computerized process measures: T-test results indicated that there were no statistically significant differences between the groups as for the number of on paper segments that were drawn (Mean MDD=29.39±8.45; Mean Controls=28.35±6.36, t₍₃₆₎=0.43 p=0.67).

Table 8 presents the means and standard deviations of the computerized process measures of the entire task. The MANOVA yielded statistically significant differences between the groups across the three measures of the entire task (F_{(3.34)}=1.38, p=0.011). As shown in Table 8, the subsequent univariate ANOVA analyses revealed that the significance was due to differences between the MDD group and controls on the mean pressure and azimuth measures but not in mean task performance time.

TABLE 8
Comparison of kinematic measures of the entire clock drawing task

| Kinematic measures of the | Mean (SD) in | Mean (SD) in |
|---|---|---|
| entire clock drawing task | MDD (n = 20) | Controls (n = 20) |
| Performance time (seconds) | 57.17 (37.23) | 43.76 (11.53) |
| Azimuth (0-360, degrees) | 129.24 (13.32) | 139.43 (11.81)* |
| Pressure (0-1024, not scaled | 535.42 (106.94) | 706.72 (134.58)** |
| units) | | |

Where: SD—standard deviation; MDD—major depressive disorder;
*Denotes a statistically significant difference between MDD and control groups at p < .05.; and
**Denotes a statistically significant difference between MDD and control groups at p < .01.

Table 9 presents the means and standard deviations of the clock drawing computerized spatial process measures per segment. The MANOVA yielded statistically significant differences between the groups across the three measures (F_{(3.32)}=3.14, p=0.039). As shown in Table 8, the subsequent univariate ANOVA analyses revealed that the significance was due to differences between the MDD group and controls on all the three measures means of segment height, width and length.

TABLE 9
Comparison of spatial kinematic measures
per segment in the clock drawing task

| Spatial kinematic measures | Mean (SD) of | Mean (SD) of |
|---|---|---|
| in the clock drawing task | MDD (n = 20) | Controls (n = 20) |
| Segments' height (in cm.) | .63 (.22) | .82 (.27)* |
| Segments' width (in cm.) | .48 (.15) | .73 (.27)** |
| Segments' length (in cm.) | 1.47 (.47) | 2.08 (.74)* |

Where: SD—standard deviation; MDD—major depressive disorder; Cm.—centimeters;
*Denotes a statistically significant difference between MDD and control groups at p < .05.; and
**Denotes a statistically significant difference between MDD and control groups at p < .01.

Correlation analysis between the clock drawing scores, the computerised measures and the depression (GDS) and cognition (MMSE) scores: No statistically significant correlations were found between the clock drawing total score and subscores (contour, numbers, hands, center) and any of the kinematic measures studied. Additionally, no statistically significant correlations were found between the clock drawing total score, the subscores of contour, hands and center, the mean number of written segments on paper, and all the spatial measures (segments' height, width and length) and the GDS/MMSE scores. However, the clock drawing numbers subscore was moderately correlated with the MMSE total score (r=0.33, p<0.05) but not with the depression state as reflected in the GDS score.

Among the entire task computerized measures, performance time was significantly correlated with the MMSE score (r=−0.46, p<0.01) but not with depression scores and azimuth was significantly correlated with the GDS score (r=−0.43, p<0.01), but not with the MMSE score. The pressure measure, however, was significantly correlated with both the GDS score (r=−0.55, p<0.01) and with the MMSE (r=0.39, p<0.01).

Discriminant analysis: In order to assess the relative importance of the different variables in differentiating between MDD and control participants, a discriminant analysis was performed. The independent variables included were the clock drawing total score, and the computerized drawing measures of the entire task (mean performance time, pressure and azimuth) and those measured per segment (width, height and length).

One discriminant function was found for group classification of all participants (Wilks' Lambda=0.508, p=0.003). The variable which made the greatest contribution to group membership was the mean pressure (loading =0.70), followed by the mean segment width (loading=−0.59), the mean segment length (loading=0.51), the mean azimuth (loading 0.41) and the mean segment height (loading 0.40). Based on this function, 81.1% of the participants overall, 89% of the MDD group, and 74% of the control group were correctly classified. A Kappa value of 0.623 (p<0.001) was calculated, demonstrating that the group classification did not occur by chance.

### Discussion

A computerized kinematic analysis of the entire clock drawing task (contour, numbers, hands, center), in elderly subjects with mild MDD and controls, demonstrated significant between group differences in most kinematic measures studied. More specifically, compared to healthy elderly persons, mean pressure and azimuth measures as well as the spatial measures of segments' height, width and length were significantly lower in the mild MDD group, while number of paper segments and performance time, did not differ.

This finding becomes more conspicuous when one considers that the scoring of the clock drawing itself, in total and of each of the components (contour, numbers, hands, center) using a traditional protocol (e.g., the Freedman method) found no differences between the groups. Given the relative “complexity” of the Freedman scoring method, and its inclusion of items sensitive to executive dysfunction (e.g., hour and minute target number indicated, hands in correct proportion), we expected to find statistically significant differences between the groups using this method, especially on the hands subscore, which however were not found. This lack of differences may be attributed to the mild degree of depression in the depressive group.

Since elderly persons in the study and in the comparison group were matched for demographics (age, sex, years of education) and controlled for the cognitive status (the MMSE score), our findings suggest that the computerized system focusing on the clock drawing task might be sensitive to altered performance among people with mild MDD. The findings corroborate the results of our previous study in a same sample of elderly subjects with mild MDD and controls that found significant between group differences for four handwriting tasks (alphabet sequence, own name and surname, filling out check, paragraph copying) across temporal, spatial and pressure kinematic measures (Rosenblum et al., 2009) whose contents are incorporated herein by reference. Although the kind of tasks in those studies were different, (writing versus drawing), the same trend was found in both studies while people with MDD decease their drawing/writing output's sizes and it was smaller in comparison to that of controls. The meaning of this decrease will be discussed below.

Regarding the entire task's four measures studied, the first two (number of written segments and performance time) considered “efficiency” measures did not differ among the groups. Given that “psychomotor retardation” is a feature of major depression (APA, 1994), we could expect the depressed participants to be slower, that is higher performance time and consequently higher mean number of written segments on paper. In fact, several studies have indicated that the function of the motor system is impaired among people with depression and that considerable slowness occurs in their voluntary muscles and fine motor skills. Other authors, however, applying kinematic analysis found that drawing was not abnormally slow in depressed subjects, while handwriting was. We may suggest that the mild nature of the MDD participants in this study might preclude these “efficiency” measures to be manifested at this stage between the groups, and that with more severe levels of depression, differences between the groups on these measures might emerge as well. Evidently both above measures in this study did not correlate with the GDS score. Indeed, only performance time correlated with the MMSE score suggesting a relation between this kinematic measure and cognitive status.

On the other hand, the mean pressure and azimuth measures for the entire task were significantly different in the two diagnostic groups. The finding that lower pressure was applied by the MDD subjects on the drawing surface while grasping the pen was also found in our previous study concerning handwriting in depression (Rosenblum et al., 2009) and described by others as well (Yanagita et al., 2006). Understandably, mean pressure was significantly correlated to depression/GDS scores. While it is conceivable that lowered hand strength or handgrip strength may be a functional measure among people with depression, this lower hand strength may also influence the mastery of the pen and following, the pen azimuth which in fact represents the motions range while drawing. In fact, the azimuth measure correlated with depression/GDS in this study, as well.

As a whole, the significantly less pressure and azimuth found in the current study means that people with depression reduce their motions range and the pressure implemented to the drawing surface. Literally, these objective representations may reflect the diminished energy for life of people with depression.

Regarding the three spatial characteristics studied, that is the segments' width, length and height, found to be significantly lower in the depressed group, similarly smaller segments were found in mild MDD in comparison to controls, especially in longer tasks, in our previous study with handwriting (Rosenblum et al., 2009). We may speculate the above spatial measures have to do with requirements from the motor system and investment of energy, both presumably diminished in depression. None of these measures, however, were correlated with either GDS or MMSE scores in this study.

The fact that none of the clock drawing scores (total and subscores) was found to be associated with any of the kinematic measures studied would raise doubts concerning a meaningful specific connection between the two, not to mention that only the clock drawing numbers' subscore correlated with the MMSE score but not with the GDS score. It may be claimed that kinematic analysis measures are sensitive in general to any drawing (or handwriting) task impairment whether in depressed or cognitively impaired subjects. We, nevertheless, may argue that these findings not only are preferable over traditional not-sensitive methods of clock drawing assessment in depression as demonstrated in this study, but would need to be considered as part of a comprehensive model of clock drawing assessment, to be suggested in our concluding remarks.

Discriminant analysis, in addition, found the above kinematic measures (mean pressure, segment width, segment length, azimuth and segment height, in descending order) as the best predictors of group membership. Overall 81% of the entire sample and 89% of the MDD group were accurately classified, which undoubtedly constitutes an impressive finding, especially given the mild nature of the depressive group. Those results indicates that it was a right decision to include also the spatial measures in the discriminate because they indeed contributes to group differentiation and well manifests MDD performance (as presented in table 9), although they were not found as significantly correlated with the MMSE and the GDS. It seems that there are benefits for documentation of actual performance measures while the person performs that are not been manifests through self report questionnaires. The MMSE indeed includes beside the self report questions also two drawing tasks however, we showed in our previous study that those two tasks were not sensitive enough to decrease in performance abilities.

Although the invention has been described in detail, nevertheless changes and modifications, which do not depart from the teachings of the present invention, will be evident to those skilled in the art. Such changes and modifications are deemed to come within the purview of the present invention and the appended claims.

### REFERENCES

Koppenhaver, K. M. (2002). Attorney's guide to document examination. Quorum books.
Koppenhaver, K. M. (2007). Forensic Document Examination: principals and practice (pp. 7-25—Principles of handwriting). NY: Humana press.
Morris, R. N. (2000). Forensic handwriting identification: Fundamental concepts and princpls. Academic press
Oh I. S. and Ching, Y. S. (2001). A class-modular feedforward neural network for handwriting recognition. Pattern recognition, 35, 229-244.
Plamondon, R., & Srihari, S. N. (2000). On-line and off-line handwriting recognition: A comprehensive survey. IEEE transactions on pattern analysis and machine intelligence, 22 (1), 63-84.
Rosenblum, S. (2008). Development, Reliability and Validity of the Teacher's Screening Questionnaire for Handwriting Proficiency (TQHP). American Journal of Occupational Therapy, 62 (3), 298-307.
Rosenblum, S. (2006). The development and standardization of the Children Activity Scales (ChAS-P/T) for the early identification of children with Developmental Coordination Disorders (DCD). Child Care Health and Development, 32, 6, 619-632, Special issue about DCD.
Wellingham-Jones P. (1991). Drugs and handwriting. PWJ pub.
Heinik, J., Werner, P., Dekel, T., Gurevitz, I., & Rosenblum, S. (2009). Computerized kinematic analysis of the clock drawing task in elderly people with mild Major Depressive Disorder: An exploratory study. Manuscript submitted to International Psychogeriatrics. (20 pp.).
Rosenblum, S., Dekel, T., Gurevitz, I., Werner, P. & Heinik, J. (in press). Handwriting process variables among elderly people with mild Major Depressive Disorder: A preliminary study. Aging: Accepted: Clinical and Experimental Research

1. A diagnosis method based on handwriting analysis, the method comprising the steps of:
(i) defining for each condition a list of characteristic indicators detectable while performing a set of predetermined handwriting tasks;
(ii) recording the handwriting tasks of a person according to the defined characteristic indicators of said condition; and
(iii) analyzing the values of the recorded indicators in order to determine whether the person is diagnosed with said condition.

2. A diagnosis method according to claim 1, wherein said condition comprises:
a personality trait, a skill, an expertise, the classification of people into appropriate professions, legal purposes, deception or truth writing, early detection of disease or dysfunction, an illness, a mental illness, a physiological condition, a mental or emotional state, refinement of certain medical diagnoses and hence prevention, identification of how drugs influence disease processes, educational assessment and intervention, or any combination thereof.

3. A diagnosis method according to claim 1, wherein the characteristic indicators comprise spatial, temporal and pressure measures for each writing stroke.

4. A diagnosis method according to claim 3, wherein the characteristic indicators comprise one or more of the following indicators: total length of writing on paper, stroke height, stroke width, stroke length, speed of writing, acceleration of writing, length of time the writing instrument stays in the air, length of time the writing instrument stays in on paper, the trajectory of the writing instrument in the air, pen tilt, azimuth, coefficient of variance, peak velocity, the pressure applied while writing or any combination thereof.

5. A diagnosis method according to claim 1, wherein analyzing the values of the recorded indicators comprises correlation between two or more recorded indicators.

6. A diagnosis method according to claim 1, wherein the list of characteristic indicators for a condition is established by giving a set of handwriting tasks to a first group known to have said condition and giving the same set of handwriting tasks to a control group known not to have said condition, and analyzing the recorded indicators of the two groups in order to establish which measured indicators characterize the first group.

7. A diagnosis method according to claim 1, wherein the list of characteristic indicators for a condition is established by giving a set of handwriting tasks to a person when the person is known to have said condition and then giving the same set of handwriting tasks to the same person when the person is known not to have said condition, and analyzing the recorded indicators of the two sets of handwriting tasks in order to establish which measured indicators characterize the person when having said condition.

8. A diagnosis method according to claim 1, wherein the handwriting tasks involves writing letters, numbers, drawings or any combination thereof.

9. A diagnosis method according to claim 1, further comprising the step of validating the handwriting analysis results with additional standardized tools.

10. A diagnosis method according to claim 1, wherein the handwriting data is collected by a digitizing tablet.

11. A diagnosis method according to claim 1, wherein the recording of the handwriting tasks of the person is done using multidimensional computerized systems.

12. A diagnosis method according to claim 1, wherein the handwriting tasks are functional, everyday tasks.

13. A diagnosis method according to claim 11, wherein the functional, everyday tasks comprise: writing own name, writing the alphabet sequence from memory and copying a text.

14. A diagnosis method according to claim 1, further comprising the step of providing the person with a self-evaluation questionnaire and integrating the responses to the questionnaire in the diagnosis of the condition.

15. A diagnosis method according to claim 14, wherein the self-evaluation questionnaire comprises questions regarding possible implications on every day performance and participation in personal, social and professional activities.

16. A diagnosis system based on handwriting analysis, comprising:
(i) a list of characteristic indicators for each condition detectable while performing a set of handwriting tasks;
(ii) a digitizer on which the handwriting is done;
(iii) a recording unit for recording the handwriting tasks of a person according to multiple indicators; and
(iv) an analyzer for analyzing the recorded indicators in order to determine whether the person is diagnosed with said condition.

17. A diagnosis system according to claim 16, wherein said condition comprises:
a personality trait, a skill, an expertise, the classification of people into appropriate professions, legal purposes, deception or truth writing, early detection of disease or dysfunction, an illness, a mental illness, a physiological condition, a mental or emotional state, refinement of certain medical diagnoses and hence prevention, identification of how drugs influence disease processes, educational assessment and intervention, or any combination thereof.

18. A diagnosis system according to claim 16, wherein the characteristic indicators comprise spatial, temporal and pressure measures for each writing stroke.

19. A diagnosis system according to claim 16, wherein the characteristic indicators comprise one or more of the following indicators: total length of writing on paper, stroke height, stroke width, stroke length, speed of writing, acceleration of writing, length of time the writing instrument stays in the air, length of time the writing instrument stays in on paper, the trajectory of the writing instrument in the air, pen tilt, azimuth, coefficient of variance, peak velocity, the pressure applied while writing or any combination thereof.

20. A diagnosis system according to claim 16, wherein the analyzer correlates between two or more recorded indicators.

21. A diagnosis system according to claim 16, wherein the list of characteristic indicators for a condition is established by giving a set of handwriting tasks to a first group known to have said condition and giving the same set of handwriting tasks to a control group known not to have said condition, and analyzing the recorded indicators of the two groups in order to establish which measured indicators characterize the first group.

22. A diagnosis system according to claim 16, wherein the list of characteristic indicators for a condition is established by giving a set of handwriting tasks to a person when the person is known to have said condition and then giving the same set of handwriting tasks to the same person when the person is known not to have said condition, and analyzing the recorded indicators of the two sets of handwriting tasks in order to establish which measured indicators characterize the person when having said condition.

23. A diagnosis system according to claim 16, wherein the handwriting tasks involves writing letters, numbers, drawings or any combination thereof.

24. A diagnosis system according to claim 16, wherein the handwriting analysis results from the analyzer are validated with additional standardized tools.

25. A diagnosis system according to claim 16, wherein the recording unit comprises one or more multidimensional computerized systems.

26. A diagnosis system according to claim 16, wherein the handwriting tasks are functional, everyday tasks.

27. A diagnosis system according to claim 16, wherein the functional, everyday tasks comprise: writing own name, writing the alphabet sequence from memory and copying a text.

28. A diagnosis system according to claim 16, wherein the person is provided with a self-evaluation questionnaire and the responses to the questionnaire are integrated in the diagnosis of the condition.

29. A diagnosis system according to claim 28, wherein the self-evaluation questionnaire comprises questions regarding possible implications on every day performance and participation in personal, social and professional activities.

30. A diagnosis system according to any of claims 16 to 29, wherein the diagnosis system is a lie detector, a graphology analyzer or a tool for identification of how drugs influence disease processes.
